(19) United States
(12) Patent Application Publication
(10) Pub. No.: US 2016/0045445 A1
(43) Pub. Date: Feb. 18, 2016

(54) COMBINATION THERAPEUTIC NANOPARTICLES

(71) Applicant: UNIVERSITY OF GEORGIA RESEARCH FOUNDATION INC., Athens, GA (US)

(72) Inventors: Shanta Dhar, Athens, GA (US); Rakesh Kumar Pathak, Athens, GA (US)

(21) Appl. No.: 14/782,618

(22) PCT Filed: Apr. 9, 2014
(86) PCT No.: PCT/US2014/033431

§ 371 Date: Oct. 6, 2015

Related U.S. Application Data
(60) Provisional application No. 61/810,076, filed on Apr. 9, 2013.

(51) Int. Cl. A61K 9/50; A61K 31/222; C07C 69/78; A61K 47/48; A61K 31/663; C07J 5/00; A61K 33/24; A61K 31/573

(52) U.S. Cl. CPC A61K 9/50; A61K 33/24; A61K 31/222; A61K 31/573; A61K 47/48246; A61K 31/663; C07J 5/0053; A61K 47/482; C07C 69/78

(57) ABSTRACT

Nanoparticles that include a chemotherapeutic agent and an anti-inflammatory are particularly cytotoxic to prostate cancer cells.

### RELATED APPLICATIONS

This application claims the benefit of priority to U.S. Provisional Patent Application No. 61/810,076, filed on Apr. 9, 2013, which application is hereby incorporated herein in its entirety to the extent that it does not conflict with the present disclosure.

### STATEMENT OF GOVERNMENTAL SUPPORT

This invention was made with government support under grant number W81XWH-12-1-0406, awarded by the Department of Defense of the United States government. The government has certain rights in the invention.

### FIELD

The present disclosure relates to nanoparticles containing therapeutic agents for therapeutic purposes such as treatment of cancer, particularly to nanoparticles containing a combination of therapeutic agents for treatment of prostate cancer, such as castration-resistance prostate cancer (CPRC).

### BACKGROUND

Prostate cancer is the second leading cause of cancer-based deaths in man in the United States. Androgen, a male sex hormone has a significant role in tumor growth of prostate cancer. Thus androgen deprivation therapy (castration) has become one of the major treatments for prostate cancer, along with some chemotherapeutics. After castration, cancer progression often diminishes significantly. However, in most cases, cancer progression eventually resumes at a stage referred as castration resistance prostate cancer (CRPC).

Castration-Resistant Prostate cancer (CRPC) is one of the most prevalent and deadly forms of cancer affecting men in the United States and around the world. Currently, chemotherapy is the only form of cancer therapy that has shown to improve the survival of those with CRPC. However, chemotherapeutic agents do not help to relieve many of the symptoms related with CRPC, such as chronic inflammation and bone metastases.

### SUMMARY

The present disclosure describes, among other things, a nanoparticle (NP) platform with the capability to deliver combinations of chemotherapeutics and one or more of anti-inflammatory agents and bone resorption inhibitors. The nanoparticles may be used for treatment of cancer. In some embodiments, the nanoparticles are used for treatment of prostate cancer. In some embodiments, the nanoparticles are used for treatment of CRPC.

As described herein, delivery of a combination of a chemotherapeutic and an anti-inflammatory agent via a nanoparticle resulted in a synergistic cytotoxic effect on prostate cancer cells. Surprisingly, delivery via a nanoparticle resulted in an improved cytotoxic effect on cancer cells relative to simultaneous administration of non-nanoparticle chemotherapeutic and anti-inflammatory agent.

In some embodiments, a method for treating prostate cancer described herein includes administering a therapeutically effective amount of a nanoparticle comprising a chemotherapeutic agent and an anti-inflammatory agent to a subject suffering from prostate cancer.

In various embodiments described herein, a nanoparticle that includes a chemotherapeutic agent and an anti-inflammatory agent is used in the manufacture of a medicament for the treatment of prostate cancer.

In some embodiments described herein, a nanoparticle includes (i) a hydrophobic nanoparticle core, wherein the hydrophobic polymer that forms at least a part of the core is selected from the group consisting of a polymer comprising polylactic acid (PLA) and a polymer comprising polylactic-co-glycolic acid (PLGA); (ii) a hydrophilic layer surrounding the core, wherein the hydrophilic polymer moiety is attached to the core via a hydrophobic polymer moiety that forms at least a part of the core; (iii) an anti-inflammatory agent attached to the core; and (iv) a chemotherapeutic agent attached to the core.

In some embodiments described herein, a method includes contacting a cancer cell with a nanoparticle that includes at least a chemotherapeutic agent and an anti-inflammatory agent. Preferably, the method includes contacting the cancer cell with an amount of the nanoparticle effective to inhibit cancerous growth of the cell. More preferably, the method includes contacting the cancer cell with a cytotoxic amount of the nanoparticle. The method may be a method of treatment of a patient with cancer, in which case the method includes administering a therapeutically effective amount of the nanoparticle to the patient. The method may further include identifying a patient suffering from, or at risk of suffering from, prostate cancer or CRPC, and administering the nanoparticle to the patient.

Advantages of one or more of the various embodiments presented herein over prior therapies and therapeutics will be readily apparent to those of skill in the art based on the following detailed description when read in conjunction with the accompanying drawings.

### BRIEF DESCRIPTION OF THE DRAWINGS

FIG. 1 is schematic representation for preparation of nanoparticles using a biodegradable polymeric platform and chemo and anti-inflammatory drugs.

FIG. 2A is a transmission electron microscope image of an embodiment of nanoparticles prepared in accordance with the teachings presented herein.

FIG. 2B is a dynamic light scattering histogram showing the size of an embodiment of nanoparticles prepared in accordance with the teachings presented herein.

FIG. 2C is a graph showing zeta potential measurements of an embodiment of nanoparticles prepared in accordance with the teachings presented herein.

FIG. 3 is a graph showing the cytotoxic profile of different constructs, including an embodiment of nanoparticles prepared in accordance with the teachings presented herein, on human prostate cancer PC-3 cells.

The schematic drawings in are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

The present disclosure describes, among other things, nanoparticles comprising combinations of chemotherapeutics and one or more of anti-inflammatory agents and bone resorption inhibitors.

Nanoparticles, as described herein, include, in some embodiments, a hydrophobic core, a hydrophilic layer surrounding the core, therapeutic agents, and one or more optional targeting moiety. In embodiments, the therapeutic agents are contained or embedded within the core. The therapeutic agents, the agents are preferably released from the core at a desired rate. In embodiments, the core is biodegradable and releases the agents as the core is degraded or eroded. The targeting moieties, if present, preferably extend outwardly from the core so that they are available for interaction with cellular components or so that they affect surface properties of the nanoparticle, which interactions or surface properties will favor preferential distribution to desired cells, such as cancer cells. The targeting moieties may be tethered to the core or components that interact with the core.

### I. CORE

The core of the nanoparticle may be formed from any suitable component or components. Preferably, the core is formed from hydrophobic components such as hydrophobic polymers or hydrophobic portions of polymers. The core may also or alternatively include block copolymers that have hydrophobic portions and hydrophilic portions that may self-assemble in an aqueous environment into particles having the hydrophobic core and a hydrophilic outer surface. In some embodiments, the core comprises one or more biodegradable polymer or a polymer having a biodegradable portion.

Any suitable synthetic or natural bioabsorbable polymers may be used. Such polymers are recognizable and identifiable by one or ordinary skill in the art. Non-limiting examples of synthetic, biodegradable polymers include: poly(amides) such as poly(amino acids) and poly(peptides); poly(esters) such as poly(lactic acid), poly(glycolic acid), poly(lactic-co-glycolic acid) (PLGA), and poly(caprolactone); poly(anhydrides); poly(orthoesters); poly(carbonates); and chemical derivatives thereof (substitutions, additions of chemical groups, for example, alkyl, alkylene, hydroxylations, oxidations, and other modifications routinely made by those skilled in the art), fibrin, fibrinogen, cellulose, starch, collagen, and hyaluronic acid, copolymers and mixtures thereof. The properties and release profiles of these and other suitable polymers are known or are readily identifiable.

In various embodiments, described herein the core comprises PLGA. PLGA is a well-known and well-studied hydrophobic biodegradable polymer used for the delivery and release of therapeutic agents at desired rates.

Preferably, the at least some of the polymers used to form the core are amphiphilic having hydrophobic portions and hydrophilic portions. The hydrophobic portions can form the core, while the hydrophilic regions may form a layer surrounding the core to help the nanoparticle evade recognition by the immune system and enhance circulation half-life. Examples of amphiphilic polymers include block copolymers having a hydrophobic block and a hydrophilic block. In some embodiments, the core is formed from hydrophobic portions of a block copolymer, a hydrophobic polymer, or combinations thereof.

The ratio of hydrophobic polymer to amphiphilic polymer may be varied to vary the size of the nanoparticle. Often, a greater ratio of hydrophobic polymer to amphiphilic polymer results in a nanoparticle having a larger diameter. Any suitable ratio of hydrophobic polymer to amphiphilic polymer may be used. In some embodiments, the nanoparticle includes about a 50/50 ratio by weight of amphiphilic polymer to hydrophobic polymer or ratio that includes more amphiphilic polymer than hydrophilic polymer, such as about 20/80 ratio, about a 30/70 ratio, about a 40/60 ratio, about a 55/45 ratio, about a 60/40 ratio, about a 65/45 ratio, about a 70/30 ratio, about a 75/35 ratio, about a 80/20 ratio, about a 85/15 ratio, about a 90/10 ratio, about a 95/5 ratio, about a 99/1 ratio, or about 100% amphiphilic polymer.

In embodiments, the hydrophobic polymer comprises PLGA, such as PLGA-COOH or PLGA-OH. In embodiments, the amphiphilic polymer comprises PLGA and PEG, such as PLGA-PEG. The amphiphilic polymer may be a dendritic polymer having branched hydrophilic portions. Branched polymers may allow for attachment of more than moiety to terminal ends of the branched hydrophilic polymer tails, as the branched polymers have more than one terminal end.

The nanoparticles described herein may have any suitable size. In some embodiments, the nanoparticles have an average diameter of about 500 nm or less, such as about 250 nm or less or about 200 nm or less. Typically, the nanoparticles will have an average diameter of about 5 nm or more. In some embodiments, the nanoparticles have an average diameter of from about 20 nm to about 300 nm, such as from about 50 nm to about 150 nm, or from about 80 nm to about 130 nm.

### II. HYDROPHILIC LAYER SURROUNDING THE CORE

The nanoparticles described herein may optionally include a hydrophilic layer surrounding the hydrophilic core. The hydrophilic layer may assist the nanoparticle in evading recognition by the immune system and may enhance circulation half-life of the nanoparticle.

As indicated above, the hydrophilic layer may be formed, in whole or in part, by a hydrophilic portion of an amphiphilic polymer, such as a block co-polymer having a hydrophobic block and a hydrophilic block.

Any suitable hydrophilic polymer or hydrophilic portion of an amphiphilic polymer may form the hydrophilic layer or portion thereof. The hydrophilic polymer or hydrophilic portion of a polymer may be a linear or branched or dendritic polymer. Examples of suitable hydrophilic polymers include polysaccharides, dextran, chitosan, hyaluronic acid, polyethylene glycol, polymethylene oxide, and the like.

In some embodiments, a hydrophilic portion of a block copolymer comprises polyethylene glycol (PEG). In embodiments, a block copolymer comprises a hydrophobic portion comprising PLGA and a hydrophilic portion comprising PEG.

A hydrophilic polymer or hydrophilic portion of a polymer may contain moieties that are charged under physiological conditions, which may be approximated by a buffered saline solution, such as a phosphate or citrate buffered saline solution, at a pH of about 7.4, or the like. In various embodiments, a hydrophilic polymer or portion of a polymer includes a hydroxyl group that can result in an oxygen anion when placed in a physiological aqueous environment. For example, the polymer may include PEG-OH where the OH serves as the charged moiety under physiological conditions.

Moieties that are charged under physiological conditions may contribute to the charge density or zeta potential of the nanoparticle. Zeta potential is a term for electro kinetic potential in colloidal systems. While zeta potential is not directly measurable, it can be experimentally determined using electrophoretic mobility, dynamic electrophoretic mobility, or the like.

A nanoparticle as described herein may have any suitable zeta potential. In various embodiments, the nanoparticles described herein have a negative zeta potential. For example, the nanoparticles may have a zeta potential of about −5 mV or less. In some embodiments, nanoparticles described herein have a zeta potential of about −15 mV; e.g., from about −17 mV to about −13 mV.

### III. THERAPEUTIC AGENTS

A nanoparticle, as described herein, may include any one or more therapeutic agent. The therapeutic agent may be embedded in, or contained within, the core of the nanoparticle. Preferably, the therapeutic agent is released from the core at a desired rate. If the core is formed from a polymer (such as PLGA) or combination of polymers having known release rates, the release rate can be readily controlled.

In embodiments, a therapeutic agent or precursor thereof is conjugated to a polymer, or other component of a nanoparticle, in a manner described above with regard to targeting moieties. The therapeutic agent may be conjugated via a cleavable linker.

The therapeutic agents may be present in the nanoparticle at any suitable concentration. For example, a therapeutic agent may be present in the nanoparticle at a concentration from about 0.01% to about 30% by weight of the nanoparticle.

In various embodiments, a nanoparticle includes one or more chemotherapeutic agent. As used herein, a “chemotherapeutic agent” is an agent for treatment of cancer, such as a cytotoxic agent or an anti-neoplastic agent. Any suitable chemotherapeutic agent may be included in a nanoparticle described herein. Examples of chemotherapeutic agents include (i) alkylating agents such as cyclophosphamide, mechlorethamine, chlorambucil, melphalan, and the like; (ii) anthracyclines such as daunorubicin, doxorubicin, epirubicin, idarubicin, mitoxantrone, valrubicin, and the like; (iii) cytoskeletal disruptors such as paclitaxel, docetaxel, and the like; (iv) epothilones such as epothilone and the like; (v) histone deactylase inhibitors such as vorinostat, romidepsin, and the like; (vi) inhibitors of topoisomerase I such as irinotecan, topotecan, and the like; (vii) inhibitors or topoisomerase II such as etoposide, teniposide, tafluposide, and the like; (viii) kinase inhibitors such as bortezomib, erlontib, gefitinib, imatinib, vermurafenib, vismodegib, vismodegib, and the like; (ix) monoclonal antibodies such as bevacizumab, cetuximab, ipilimuman, ofatumumab, ocrelizumab, panitumab, rituximab, and the like; (x) nucleotide analogs and precursor analogs such as azacitidine, azathioprine, capecitabine, cytarabine, doxifluridine, fluorouracil, gemcitabine, hydroxyurea, mercaptopurine, methotrexate, tioguanine, and the like; (xi) peptide antibiotics such as bleomycin, actinomycin, and the like; (xii) platinum-based agents such as carboplatin, cisplatin, oxaliplatin, and the like; (xiii) retinoids such as tretinoin, alitretinoin, bexarotene, and the like; (xiv) vinca alkaloids and derivatives such as vinblastine, vincristine, cindesine, vinorelbine, and the like; (xv) and the like. In some embodiments, at least one of the one or more chemotherapeutics are selected from the group consisting of docetaxel, mitoxantrone, paclitaxel, satraplatin, and cisplatin.

In various embodiments, a nanoparticle includes one or more anti-inflammatory agents. Any suitable anti-inflammatory agent may be included in a nanoparticle described herein. Examples of anti-inflammatory agents that may be used include (i) corticosteroids such as prednisone, prednislone, dexamethasone, fludrocortisone, hydrocortisone, and the like; (ii) non-steroidal anti-inflammatory drugs (NSAIDs) such as aspirin, choline and magnesium salicylates, choline salicylate, celecoxib, diclofenac potassium, diclofenac sodium, diclofenac sodium with misoprostol, diflunisal, etodolac, fenoprofen calcium, flurbiprofen, ibuprofen, indomethacin, ketoprofen, magnesium salicylate, meclofenamate sodium, mefenamic acid, meloxicam, nabumetone, naproxen, aaproxen sodium, oxaprozin, piroxican, rofecoxib, salsalate, sodium salicylate, sulindac, tolmetin sodium, valdecoxib, and the like; (iii) TNF-alpha inhibitors (soluble receptors, antibodies, xanthine derivates, 5-HT2A agonists, etc.) such as infliximab, adalimumab, certolizumab pegol, golimumab, etanercept, pentoxifylline, bupropion, (R)-DOI, TCB-2, LSD, LA-SS-Az, and the like; (iv) and the like. In various embodiments, the anti-inflammatory agent is a NSAID. In some embodiments, the NSAID is aspirin.

In various embodiments, a nanoparticle includes one or more inhibitor of bone resorption. PCa bone metastases are predominantly osteoblastic, with abnormal deposition of unstructured bone accompanied by increased skeletal fractures, spinal cord compression, and severe bone pain. Chemotherapy improves survival but has limited impact on bone metastases in CRPC. Bone-targeted therapies do reduce skeletal-related events (SREs) but have not shown an impact on survival in most patients. Thus, bone metastases represent a clinical challenge with limited available solutions. Bone metastases associated with markedly increased rates of bone formation and resorption provides a rationale for use of bone resorption inhibitors in a single formulation in combination with chemotherapeutic and anti-inflammatory agents to provide CRPC patients relieve from bone pain. Any suitable inhibitor of bone resorption may be included in a nanoparticle described herein. Examples of inhibitors of bone resorption that may be used include (i) bisphosphonates such as alendronate, cholecalciferol, zoledronic acid, etidronate, ibandronate, risedronate, zoledronic acid, pamidronate, tiludronate, and the like; (ii) other inhibitors of bone resorption such as gallium nitrate, denosumab, and the like.

In some embodiments, the nanoparticle includes a chemotherapeutic agent and an anti-inflammatory agent. In various embodiments, the nanoparticle includes a chemotherapeutic agent, an anti-inflammatory agent and an inhibitor of bone resorption.

In various embodiments, the nanoparticle includes one or more prodrugs of therapeutic agents, where the prodrugs are conjugated to a polymer, another therapeutic agent, or the like and a when released are therapeutic agents. For purposes of the present disclosure a conjugated therapeutic agent and a prodrug are used interchangeably. In addition, for the purposes of the present disclosure, a therapeutic agent referenced regarding a nanoparticle is the therapeutic agent that is released from the nanoparticle. For example, a nanoparticle containing a cisplatin prodrug or an aspirin prodrug, where the prodrug converts to cisplatin or aspirin upon release from the nanoparticle is referred to herein as a nanoparticle that includes cisplatin and aspirin.

In embodiments, one or more therapeutic agents are conjugated to a polymer that forms a part of the nanoparticle. For purposes of example and proof of concept, PLA having conjugated aspirin and cisplatin was synthesized for incorporation into nanoparticles. The anti-inflammatory drug, aspirin functionalized alkyne-[G-2] Bis-MPA dendron was synthesized as shown in Scheme 1. PLA bearing azide functionality in one end and aliphatic —OH functionality in another end was synthesized to incorporate chemotherapeutic, Pt(IV) prodrugs (Scheme 2). Finally the click chemistry approach was utilized to combine these two constructs to give a PLA polymer functionalized with both the drugs (Scheme 3).

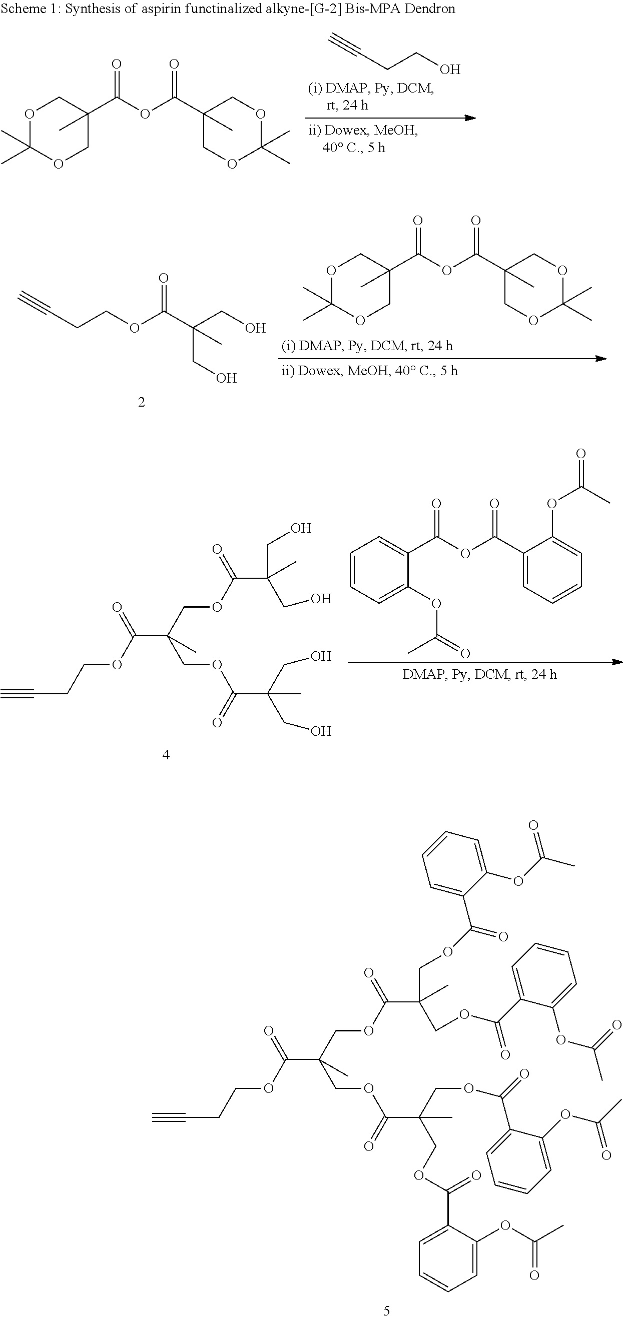

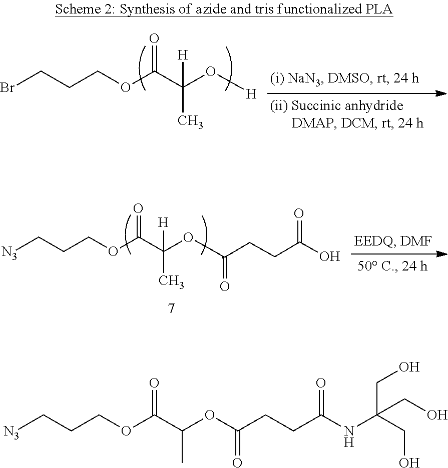

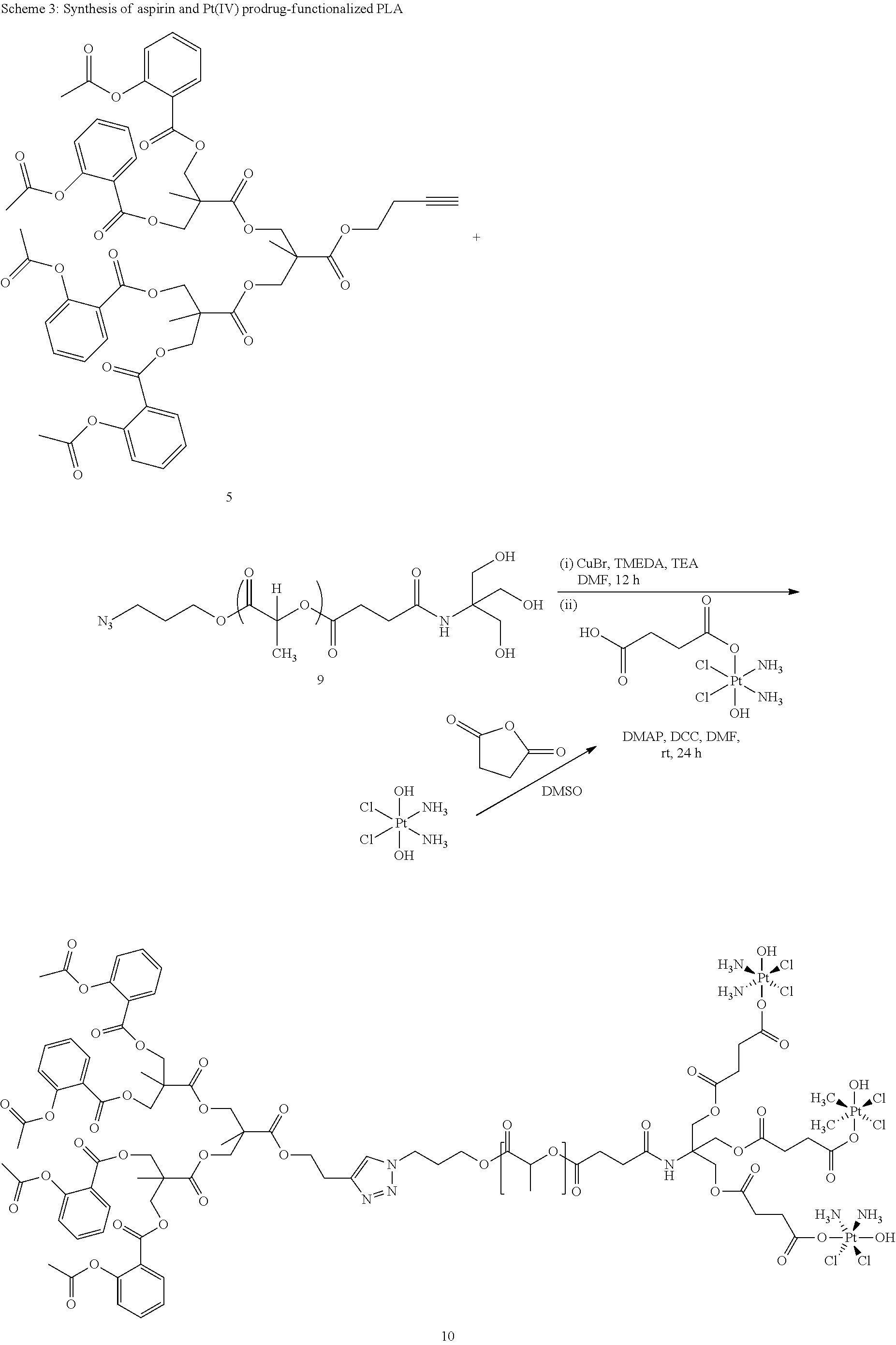

By way of further example, PLGA having conjugated aspirin and cisplatin was synthesized for incorporation into nanoparticles as illustrated below in Scheme 4. In order to achieve high reproducibility and better loading efficiencies of the drugs to the polymeric platform, PLGA was functionalized with Tris molecule bearing three sites to attach drugs covalently. Individual drugs were attached to using simple ester linkage to the PLGA-Tris platform.

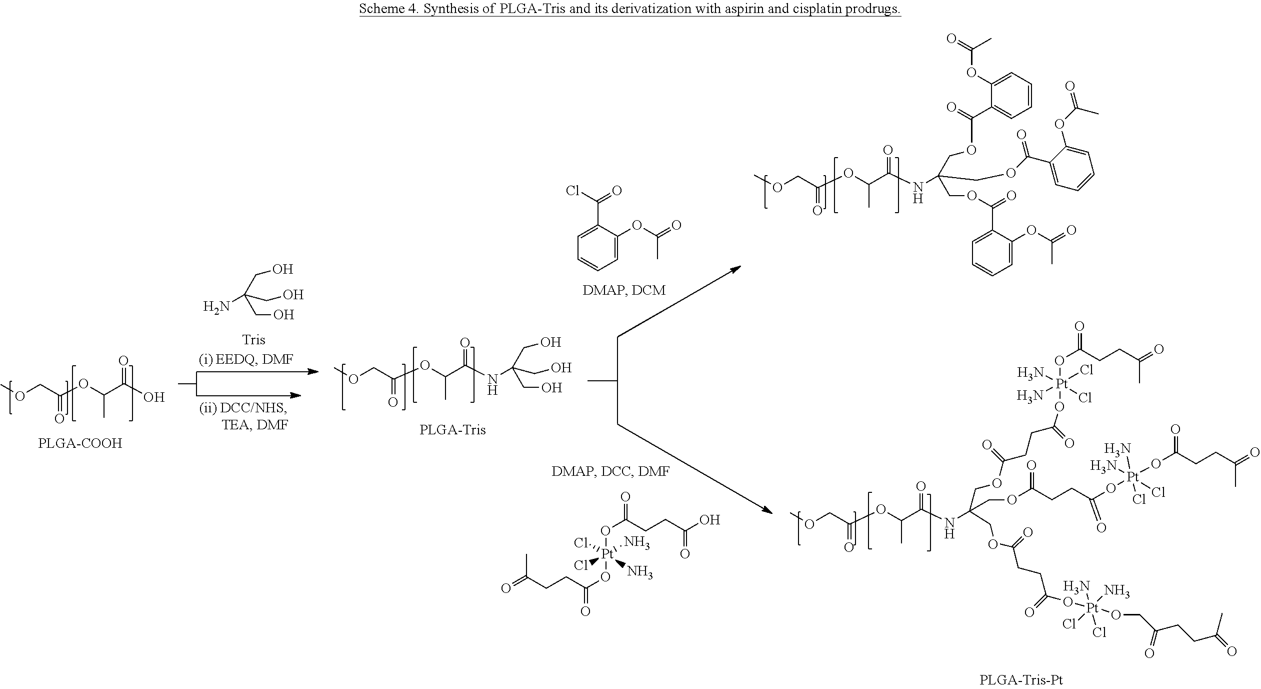

By way of yet another example, PLGA-conjugated to the bone metastasis inhibitor pamidronate was synthesized according to Scheme 5.

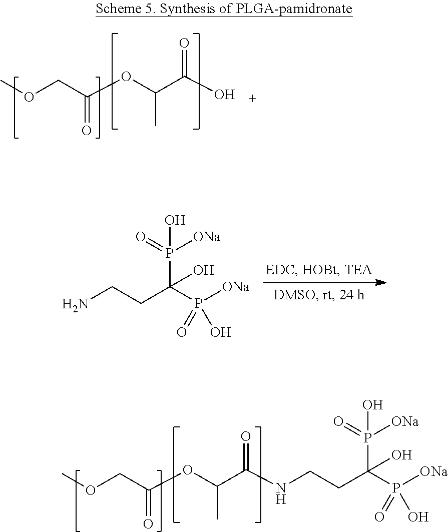

By way of yet another example, a prednisone-conjugated dendron was synthesized according to Scheme 6, in which prednisone was functionalized with succinate moiety to attach it on dendron surface by ester linkage. This alkyne derived prednisone decorated Dendron can be utilized to attach to a polymer backbone along with, for example, the cisplatin drug.

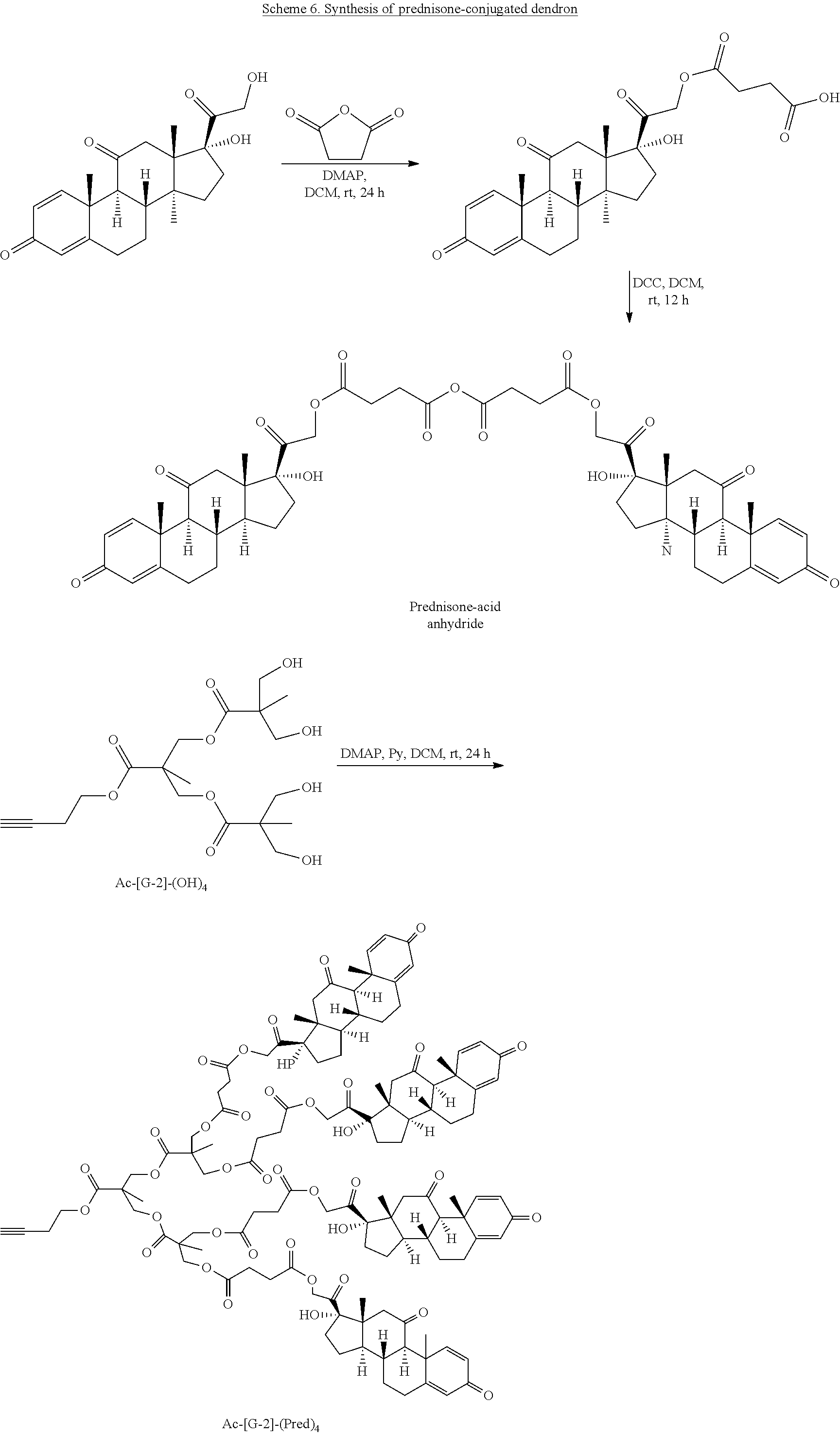

It will be understood that other therapeutic agents may be synthesized or modified for attachment to a polymer and that aspirin functionalized alkyne-[G-2] Bis-MPA Dendron and Pt(IV) prodrugs are presented as examples.

By way of example, synthesis schemes described above and in Pathak R. K., et al. (2014 Feb. 10), The prodrug platin-A: simultaneous release of cisplatin and aspirin. Agnew Chem. Int. Ed. Engl.; 53(7):1963-7 may be combined where an aspirin prodrug may be conjugated to Pt(IV) (e.g., as described in Parhak et al.) and PLA or other suitable polymer may be also be conjugated to Pt(IV) (e.g., as described above).

### IV. CANCER TARGETING MOIETIES

Nanoparticles described herein may optionally include one or more moieties that target the nanoparticles to cancer cells. As used herein, “targeting” a nanoparticle to a cancer cell means that the nanoparticle accumulates in the targeted cancer relative to other cells at a greater concentration than a substantially similar non-targeted nanoparticle. A substantially similar non-targeted nanoparticle includes the same components in substantially the same relative concentration (e.g., within about 5%) as the targeted nanoparticle, but lacks a targeting moiety.

The cancer targeting moieties may be tethered to the core in any suitable manner, such as binding to a molecule that forms part of the core or to a molecule that is bound to the core. In some embodiments, a targeting moiety is bound to a hydrophilic polymer that is bound to a hydrophobic polymer that forms part of the core. In various embodiments, a targeting moiety is bound to a hydrophilic portion of a block copolymer having a hydrophobic block that forms part of the core.

The targeting moieties may be bound to any suitable portion of a polymer. In some embodiments, the targeting moieties are attached to a terminal end of a polymer. In various embodiments, the targeting moieties are bound to the backbone of the polymer, or a molecule attached to the backbone, at a location other than a terminal end of the polymer. More than one targeting moiety may be bound to a given polymer. In embodiments, the polymer is a dendritic polymer having multiple terminal ends and the targeting moieties may be bound to more than one of terminal ends.

The polymers, or portions thereof, to which the targeting moieties are bound may contain, or be modified to contain, appropriate functional groups, such as —OH, —COOH, —NH₂, —SH, —N₃, —Br, —Cl, —I, —CH═CH₂, CCH, —CHO or the like, for reaction with and binding to the targeting moieties that have, or are modified to have, suitable functional groups.

Targeting moieties may be present in the nanoparticles at any suitable concentration. It will be understood that the concentration may readily be varied based on initial in vitro analysis to optimize prior to in vivo study or use. In some embodiments, the targeting moieties will have surface coverage of from about 5% to about 100%.

Preferably, a targeting moiety is attached to a hydrophilic polymer or hydrophilic portion of a polymer so that the targeting moiety will extend from the core of the nanoparticle to facilitate the effect of the targeting moiety. In various embodiments, a targeting moiety is attached to PEG.

Any suitable cancer targeting moiety may be attached to a nanoparticle described herein. Examples of cancer targeting moieties include moieties that bind cell surface antigens or markers that are selective to cancer cells or over-expressed, up-regulated or otherwise present in amounts not found in non-cancer cells. In some embodiments, the cancer targeting moiety is a prostate cancer targeting moiety. Examples of prostate cancer targeting moieties include moieties that selectively bind to prostate specific membrane antigen (PSMA), such as an antibody or antibody fragment that binds PSMA, a peptide having an amino acid sequence of WQPDTAHHWATL (SEQ ID NO:1) or a PSMA binding fragment thereof, and the like.

### V. SYNTHESIS OF NANOPARTICLE

Nanoparticles, as described herein, may be synthesized or assembled via any suitable process. Preferably, the nanoparticles are assembled in a single step to minimize process variation. A single step process may include nanoprecipitation and self-assembly.

In general, the nanoparticles may be synthesized or assembled by dissolving or suspending hydrophobic components in an organic solvent, preferably a solvent that is miscible in an aqueous solvent used for precipitation. In embodiments, acetonitrile is used as the organic solvent, but any suitable solvent such as dimethlyformamide (DMF), dimethyl sulfoxide (DMSO), acetone, or the like may be used. Hydrophilic components are dissolved in a suitable aqueous solvent, such as water, 4 wt-% ethanol, or the like. The organic phase solution may be added drop wise to the aqueous phase solution to nanoprecipitate the hydrophobic components and allow self-assembly of the nanoparticle in the aqueous solvent.

A process for determining appropriate conditions for forming the nanoparticles may be as follows. Briefly, functionalized polymers and other components, if included or as appropriate, may be co-dissolved in organic solvent mixtures. This solution may be added drop wise into hot (e.g., 65° C.) aqueous solvent (e.g., water, 4 wt-% ethanol, etc.), whereupon the solvents will evaporate, producing nanoparticles with a hydrophobic core surrounded by a hydrophilic polymer component, such as PEG. Once a set of conditions where a desired level of targeting moiety surface loading (if present) has been achieved, therapeutic agents may be included in the nanoprecipitation and self-assembly of the nanoparticles.

If results are not desirably reproducible by manual mixing, microfluidic channels may be used.

Nanoparticles may be characterized for their size, charge, stability, drug loading, drug release kinetics, surface morphology, and stability using well-known or published methods.

Nanoparticle properties may be controlled by (a) controlling the composition of the polymer solution, and (b) controlling mixing conditions such as mixing time, temperature, and ratio of water to organic solvent. The likelihood of variation in nanoparticle properties increases with the number of processing steps required for synthesis.

The size of the nanoparticle produced can be varied by altering the ratio of hydrophobic core components to amphiphilic shell components. Nanoparticle size can also be controlled by changing the polymer length, by changing the mixing time, and by adjusting the ratio of organic to the phase. Prior experience with nanoparticles from PLGA-b-PEG of different lengths suggests that nanoparticle size will increase from a minimum of about 20 nm for short polymers (e.g. PLGA₃₀₀₀-PEG₇₅₀) to a maximum of about 150 nm for long polymers (e.g. PLGA_{100,000}-PEG_{10,000}). Thus, molecular weight of the polymer will serve to adjust the size.

Nanoparticle surface charge can be controlled by mixing polymers with appropriately charged end groups. Additionally, the composition and surface chemistry can be controlled by mixing polymers with different hydrophilic polymer lengths, branched hydrophilic polymers, or by adding hydrophobic polymers.

Once formed, the nanoparticles may be collected and washed via centrifugation, centrifugal ultrafiltration, or the like. If aggregation occurs, nanoparticles can be purified by dialysis, can be purified by longer centrifugation at slower speeds, can be purified with the use surfactant, or the like.

Once collected, any remaining solvent may be removed and the particles may be dried, which should aid in minimizing any premature breakdown or release of components. The nanoparticles may be freeze dried with the use of bulking agents such as mannitol, or otherwise prepared for storage prior to use.

It will be understood that therapeutic agents may be placed in the organic phase or aqueous phase according to their solubility.

Nanoparticles described herein may include any other suitable components, such as phospholipids or cholesterol components, generally know or understood in the art as being suitable for inclusion in nanoparticles. Copending patent application, PCT/US2012/053307, describes a number of additional components that may be included in nanoparticles. Copending patent application, PCT/US2012/053307.

Nanoparticles disclosed in PCT/US2012/053307 include targeting moieties that target the nanoparticles to apoptotic cells, such as moieties that target phosphatidylserine (PS). The targeting moieties are conjugated to a component of the nanoparticle. Such moieties include various polypeptides or zinc 2,2′-dipicolylamine (Zn²⁺-DPA) coordination complexes. In embodiments, the nanoparticles described herein are free or substantially fee of apoptotic cell targeting moieties. In embodiments, the nanoparticles described herein are free or substantially fee of apoptotic cell targeting moieties that are conjugated to a component of the nanoparticle. In embodiments, the nanoparticles described herein are free or substantially fee of PS targeting moieties. In embodiments, the nanoparticles described herein are free or substantially fee of PS targeting moieties that are conjugated to a component of the nanoparticle. In embodiments, the nanoparticles described herein are free or substantially fee of PS-polypeptide targeting moieties or Zn²⁺-DPA moieties. In embodiments, the nanoparticles described herein are free or substantially fee of PS-polypeptide targeting moieties or Zn²⁺-DPA moieties that are conjugated to a component of the nanoparticle.

Nanoparticles disclosed in PCT/US2012/053307 include macrophage targeting moieties, such as simple sugars, conjugated to components of the nanoparticles. In embodiments, the nanoparticles described herein are free or substantially free of macrophage targeting moieties. In embodiments, the nanoparticles described herein are free or substantially free of macrophage targeting moieties that are conjugated to the nanoparticle or a component thereof. In embodiments, the nanoparticles described herein are free or substantially free of simple sugar moieties. In embodiments, the nanoparticles described herein are free or substantially free of simple sugar moieties that are conjugated to the nanoparticle or a component thereof.

### VI. USE AND TESTING

In general, a nanoparticle as described herein may be used for treatment or study of cancer, such as prostate cancer; more particularly castrate resistant prostate cancer.

The performance and characteristics of nanoparticles produced herein may be tested or studied in any suitable manner. By way of example, therapeutic efficacy can be evaluated using cell-based assays. Toxicity, bio-distribution, pharmacokinetics, and efficacy studies can be tested in cells or rodents or other mammals. Zebrafish or other animal models may be employed for therapy studies. Rodents, rabbits, pigs, or the like may be used to evaluate therapeutic potential of nanoparticles. Some additional details of studies that may be performed to evaluate the performance or characteristics of the nanoparticles, which may be used for purposes of optimizing the properties of the nanoparticles are described below. However, one of skill in the art will understand that other assays and procedures may be readily performed.

Uptake and binding characteristics of nanoparticles may be evaluated in any suitable cell line, such as RAW 264.7, J774, jurkat, and HUVECs cells. The immunomodulatory role of nanoparticles may be assayed by determining the release of cytokines when these cells are exposed to varying concentrations of nanoparticles. Complement activation may be studied to identify which pathways are triggered using columns to isolate opsonized nanoparticles; e.g. as described in Salvador-Morales C, Zhang L, Langer R, Farokhzad O C, Immunocompatibility properties of lipid-polymer hybrid nanoparticles with heterogeneous surface functional groups, Biomaterials 30: 2231-2240, (2009). Because nanoparticle size can be a factor that determines biodistribution, nanoparticles may be binned into various sizes (e.g., 20-40, 40-60, 60-80, 80-100, 100-150, and 150-300 nm) and tested according to size.

Any cell type appropriate for a therapeutic agent employed in a nanoparticle may be used to evaluate therapeutic efficacy or proper targeting. Assays appropriate for the therapeutic or pharmacologic outcome may be employed, as are generally understood or known in the art.

Biodistribution (bioD) and pharmacokinetic (PK) studies may be carried out in rats or other suitable mammals. For PK and bioD analysis, Sprague Dawley rats may be dosed nanoparticles through a lateral tail vein injection. The bioD may be followed initially for 1-24 h after injection. Animals may be sacrificed; and brain, heart, intestine, liver, spleen, kidney, muscle, bone, lung, lymph nodes, gut, and skin may be excised, weighed, and homogenized. Tissue concentration and blood half-life determinations may be made at various time points

Therapeutic dosages of nanoparticles effective for human use can be estimated from animal studies according to well-known techniques, such as surface area or weight based scaling.

### VII. DEFINITIONS

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

As used herein, “have”, “having”, “include”, “including”, “comprise”, “comprising” or the like are used in their open ended sense, and generally mean “including, but not limited to”. It will be understood that “consisting essentially of”, “consisting of”, and the like are subsumed in “comprising” and the like.

As used herein, “disease” means a condition of a living being or one or more of its parts that impairs normal functioning. As used herein, the term disease encompasses terms such disease, disorder, condition, dysfunction and the like.

As used herein, “treat” or the like means to cure, prevent, or ameliorate one or more symptom of a disease.

As used herein, a compound that is “hydrophobic” is a compound that is insoluble in water or has solubility in water below 1 milligram/liter.

As used herein a compound that is “hydrophilic” is a compound that is water soluble or has solubility in water above 1 milligram/liter.

As used herein, “bind,” “bound,” or the like means that chemical entities are joined by any suitable type of bond, such as a covalent bond, an ionic bond, a hydrogen bond, van der walls forces, or the like. “Bind,” “bound,” and the like are used interchangeable herein with “attach,” “attached,” and the like.

As used herein, a molecule or moiety “attached” to a core of a nanoparticle may be embedded in the core, contained within the core, attached to a molecule that forms at least a portion of the core, attached to a molecule attached to the core, or directly attached to the core.

As used herein, a “comparable” dose of a free therapeutic agent relative to the agent in a nanoparticle is a dose of the free therapeutic that contains essentially the same amount of therapeutic agent as in the dose of the nanoparticle.

As used herein, a “free” therapeutic agent is a therapeutic agent that is not in or associated with a nanoparticle

### VIII. INCORPORATION BY REFERENCE

Each of the patents, published patent applications, and non-patent literature cited herein is hereby incorporated herein by reference in its respective entirety to the extent that it does not conflict with the present disclosure.

In the following, non-limiting examples are presented, which describe various embodiments of representative nanoparticles, methods for producing the nanoparticles, and methods for using the nanoparticles.

### EXAMPLES

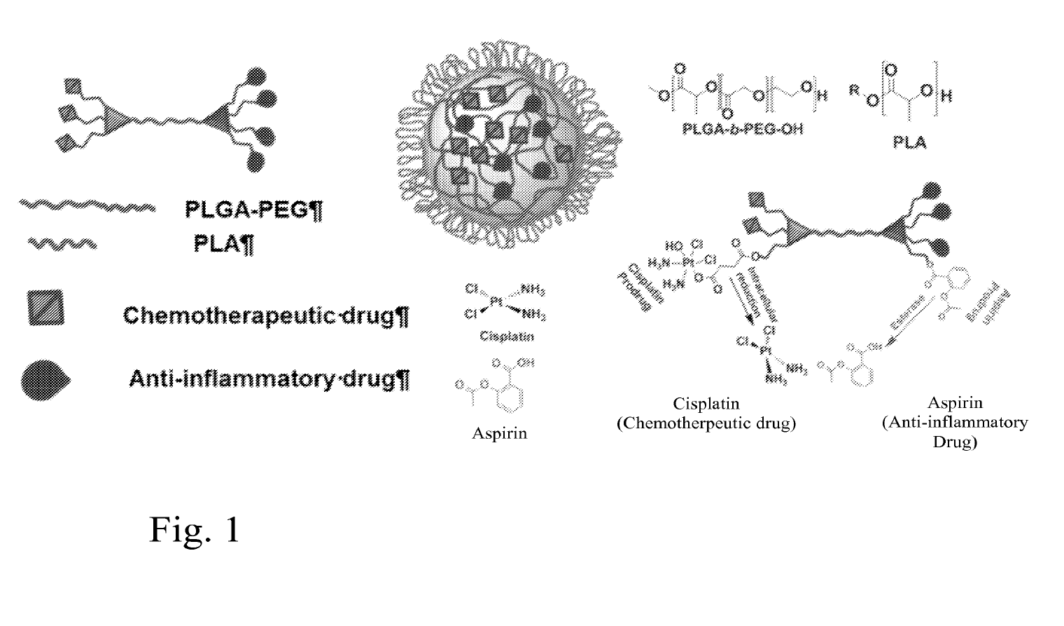

As proof of concept of the use of nanoparticles to deliver chemotherapeutic agents and anti-inflammatory agents for treatment of cancer, we synthesized and characterized an anti-inflammatory agent, aspirin, and a chemotherapeutic, Pt(IV) prodrug bearing dendron/branched terminal polymers (bow-tie type polymers) and assembled these polymers to construct combination therapeutic nanoparticles. FIG. 1 illustrates the general scheme for construction of the nanoparticles. These nanoparticles were screened for their anticancer activity using prostate cancer PC-3 cells.

Aspirin functionalized alkyne-[G-2] Bis-MPA dendron was synthesized and characterized as shown in Scheme 1 above. Polylactide (PLA) bearing azide functionality in one end and aliphatic —OH functionality in another end was synthesized to incorporate chemotherapeutic, Pt(IV) prodrugs (Scheme 2, above). Finally the click chemistry approach was utilized to combine these two constructs to give a PLA polymer functionalized with both the drugs (Scheme 3, above). These compounds were characterized by different analytical and spectroscopic techniques.

Synthesis and Characterization of 1:

Butyne alcohol (0.68 g, 9.7 mmol) and DMAP (0.18 g, 1.5 mmol) were dissolved in pyridine (2.3 g, 2.92 mmol) in a 50 mL round bottom flask, followed by the addition of 15 mL CH₂Cl₂. The anhydride of isopropylidene-2,2-bis(methoxy)propionic acid (Bis-MPA) (4.18 g, 12.6 mmol) was added slowly. The solution was stirred at room temperature for 12 h. The reaction was quenched with 3 mL of water under vigorous stirring, followed by dilution with 50 mL of CH₂Cl₂ and the solution was washed with and 10% of Na₂CO₃ (3×20 mL) and brine (10 mL). The organic phase was dried with MgSO4, filtered, and concentrated. The crude product was purified by flash chromatography on silica, eluting with hexane (100 mL) and gradually increasing the polarity to EtOAc:hexane (10:90, 700 mL), followed by EtOAc:hexane (15:85) to give 1 as a colorless oil. Yield: 1.98 g (89%). ¹H NMR (CDCl₃, 400 MHz): δ PPM, 4.24 (t, 2H), 4.20 (d, 2H), 3.63 (d, 2H), 2.54 (t, 2H), 1.98 (t, 1H), 1.42 (s, 3H), 1.38 (s, 3H), 1.20 (s, 3H). ¹³C NMR (CDCl₃, 300 MHz): δ 174, 98.0, 79.7, 69.85, 65.90, 62.23, 4186, 24.19, 23.06, 18.90.

Synthesis and Characterization of 2:

DOWEX 50W resin (3.5 g) was added to a solution of 1, (1.73 g, 7.64 mmol) in 50 mL of methanol in a 100 mL round bottom flask. The mixture was stirred at 40° C. The resin was filtered off and the filtrate was concentrated and dried under high vacuum to give 2, as colorless oil. Yield: 1.26 g (89%). ¹H NMR (CDCl₃ 400 MHz) δ: 1.07 (s, 3H), 2.03 (t, 1H), 2.57 (t, 2H), 3.73 (d, 2H), 3.87 (d, 2H), 4.26 (d, 2H). ¹³C NMR (CDCl₃, 300 MHz) δ: 18.91, 17.06, 49.38, 62.35, 67.76, 70.12, 79.94, 175.45.

Synthesis and Characterization of 3:

Compound 2 (1.00 g, 5.37 mmol) and DMAP (0.197 g, 1.611 mmol) were dissolved in pyridine (3.5 mL, 42.96 mmol) in a 100 mL round bottom flask, followed by the addition of 15 mL CH₂Cl₂. The anhydride of isopropylidene-2,2-bis(methoxy)propionic acid (Bis-MPA) (4.60 g, 13.96 mmol) was added slowly. The solution was stirred at room temperature overnight. The reaction was quenched with 3 mL of water under vigorous stirring, followed by dilution with 50 ml of CH₂Cl₂ and the solution was washed with and 10% of aq. Na₂CO₃ (3×20 mL), 10% of aq. NaHSO₄ (3×20 mL) and brine (10 mL). The organic phase was dried with MgSO₄, filtered, and concentrated. The crude product was purified by flash chromatography on silica, eluting with hexane (100 mL) and gradually increasing the polarity to EtOAc:hexane (10:90, 700 mL), followed by EtOAc:hexane (15:85) to give 3 as a colorless oil. Yield: 1.37 g (53%). ¹H NMR (CDCl₃, 400 MHz): δ PPM, 4.19 (br s, 4H), 4.10 (t, 2H), 4.38 (d, 2H), 3.49 (d, 2H), 2.40 (t, 2H), 2.03 (br t, 1H), 1.27 (s, 6H), 1.22 (s, 6H), 1.16 (s, 3H), 1.06 (s, 6H). ¹³C NMR (CDCl₃, 300 MHz): δ 173.48, 172.26, 98.08, 79.7, 70.13, 65.94, 65.91, 65.32, 62.71, 46.75, 42.03, 24.93, 22.23, 18.83, 18.53, 17.65.

Synthesis and Characterization of 4:

7.5 g of DOWEX 50W resin was added to a solution of 3, (1.32 g, 2.66 mmol) in 50 mL of methanol in a 100 mL round bottom flask. The mixture was stirred at 40° C. The resin was filtered off and the filtrate was concentrated and dried under high vacuum to give 2, as colorless oil. Yield: 1.38 g. ¹H NMR (CDCl₃ 400 MHz) δ: 1.05 (s, 3H), 129 (s, 3H), 2.02 (t, 1H), 2.54 (t, 2H), 3.01 (br s, 4H), 3.65-3.78 (m, 8H), 4.23 (t, 2H), 4.41 (d, 2H). ¹³C NMR (CDCl₃, 300 MHz) δ: 175.08, 172.79, 79.57, 70.30, 67.08, 64.81, 62.89, 49.76, 46.35, 18.83, 18.06, 17.11.

Synthesis and Characterization of 5:

Compound 4 (0.2 g, 0.46 mmol) and DMAP (0.033 g, 0.276 mmol) were dissolved in pyridine (0.600 mL, 7.36 mmol) in a 100 mL round bottom flask, followed by the addition of 15 mL CH₂Cl₂. The anhydride of aspirin (0.819 g, 2.396 mmol) was added slowly. The solution was stirred at room temperature overnight. The reaction was quenched with 3 mL of water under vigorous stirring, followed by dilution with 50 ml of CH₂Cl₂ and the solution was washed with and 10% of aq. Na₂CO₃ (3×20 mL), 10% of aq. NaHSO₄ (3×20 mL) and brine (10 mL). The organic phase was dried with MgSO4, filtered, and concentrated. The crude product was purified by flash chromatography on silica, eluting EtOAc:hexane (15:85) to give 15 as a colorless oil. Yield: 1.37 g (53%). ¹H NMR (CDCl₃, 400 MHz): δ PPM, 7.06-8.19 (m, 16H), 4.17-4.44 (m, 12H), 1.97-2.46 (m, 14H), 1.20 (br s, 16H), 0.85 (br s, 8H). ¹³C NMR (CDCl₃, 300 MHz): δ 171.93, 170.43, 169.55, 150.98, 134.50, 132.51, 131.38, 126.05, 123.81, 70.21, 65.79, 62.84, 53.41, 46.62, 34.65, 29.04, 25.26, 20.98, 18.79, 17.12, 14.09. ESI MS: 1089.3 (M+Na)+.

Synthesis and Characterization of 6, N₃-PLA-OH:

Br-PLA-OH (2.37 g, 0.25 mmol) and sodium azide (578 mg, 8.88 mmol) was mixed in DMSO. This reaction mixture was heated to 50° C. for 24 h. Reaction mixture was cooled to room temperature and then poured in the chilled water followed by the quick extraction of the polymer in the dichloromethane. Solvent was dried over MgSO₄ and concentrated under diminished pressure to get viscous oil. This was then recrystallized using diethylether to get white solid product. Yield 1.98 g. ¹H NMR (CDCl₃, 400 MHz): δ ppm 5.17 (q, 171), 4.23 (t, 2H), 3.38 (t, 2H), 1.91 (t, 2H), 1.58 (d, 532H). ¹³C NMR (CDCl₃): 169.54, 68.97, 66.67, 47.84, 28.10, 16.61. IR 0.3512, 3003, 2949, 2100, 1753, 1454, 1378, 1080, 878, 754.

Synthesis and Characterization of 7, N₃-PLA-COOH:

N₃-PLA-OH (2.0 g, 0.25 mmol), DMAP (153 mg, 1.25 mmol) and succinic anhydride (625 mg, 6.25 mmol) were dissolved in 30 mL of dichloromethane and left to react overnight under stirring at rt. Product was isolated by multiple precipitations using dichloromethane-methanol (1:1)—diethyl ether (excess) solvents. This was dried under diminished pressure to get white solid product. Yield 1.90 g. ¹H NMR (CDCl₃, 400 MHz): δ PPM, 5.14 (t, 140H) 4.22 (t, 2H), 3.37 (t, 2H), 2.70 (m, 4H), 1.90 (t, 2H), 1.58 (d, 446H). ¹³C NMR (CDCl₃, 100 MHz): δ 175.15, 65.06, 31.90, 29.68, 29.64, 29.56, 29.49, 29.34, 29.22, 28.54, 25.85, 22.67, 14.09. Yield 1.98 g. ¹H NMR (CDCl₃, 400 MHz): δ ppm 5.17 (q, 171), 4.23 (t, 2H), 3.38 (t, 2H), 1.91 (t, 2H), 1.58 (d, 532H). ¹³C NMR (CDCl₃): 169.54, 68.97, 66.67, 47.84, 28.10, 16.61.

Synthesis and characterization of 8, N3-PLA-Tris:

N₃-PLA-COOH (0.745 g, 0.0919 mmol), Tris (0.113 g, 0.919 mmol) and EEDQ (0.230 g, 0.919 mmol) were dissolved in 30 mL of dimethylformamide. This mixture was stirred at 60° C. for 24 h followed by drying under vacuum. The crude mixture was purified by dialysis (MWCO 2000) for 24 h against 3 L water which was changed every 12 h or precipitated with dichloromethane:methanol:diethylether (1:4:5). The resultant residue was frozen in liquid nitrogen and lyophilized. Yield (0.500 g, 72%). ¹H NMR (CDCl₃, 400 MHz): δ PPM, 5.14 (t, 140H) 4.34 (br s, 3H), 4.18 (br t, 2H), 3.67 (br 6H), 3.36 (t, 2H), 2.67 (m, 4H), 1.88 (t, 2H), 1.55 (d, 485H). ¹³C NMR (CDCl₃, 100 MHz): δ 169.54, 68.97, 66.56, 16.61.

Synthesis and Characterization of 9, Asp-PLA-Tris:

N₃-PLA-Tris (46 mg, 0.0057 mmol), Ac-[G-2]-(Asp)₄ (12.27 mg, 0.0115 mmol), CuBr (2.56 mg, 0.0115) and PMDETA (1.99 mg, 0.0115 mmol) were dissolved in 5 mL of dimethylformamide under nitrogen purging. This mixture was stirred at rt under bubbling of nitrogen gas. Solvent was evaporated to dryness. The crude mixture was purified by precipitating with dichloromethane:methanol:diethyl ether (1:4:5) mixture. Resultant pellet was frozen in liquid nitrogen and lyophilized. Yield (41 mg, 71%). ¹H NMR (CDCl₃, 400 MHz): δ PPM, 7.91 (d, 4H), 7.55 (t, 4H), 7.29 (t, 4H), 7.09 (d, 4H), 5.14 (t, 89H) 4.43-4.15 (m, 16H), 3.66 (br 6H), 3.37 (t, 2H), 2.47 (m, 4H), 2.34 (s, 12H), 2.03 (d, 9H), 1.89 (t, 2H), 1.56 (d, 305H). ¹³C NMR (CDCl₃, 100 MHz): δ 171.66, 169.52, 163.29, 150.76, 134.00, 131.34, 125.85, 123.64, 122.40, 69.22, 65.55, 62.88, 61.63, 47.37, 46.11, 20.74, 16.59.

Synthesis and Characterization of 10, Asp-PLA-Tris-Pt:

Asp-PLA-Tris (35 mg, 0.0036 mmol), monosuccinato-Pt(IV) (13 mg, 0.029 mmol), EDC (5.6 mg, 0.029 mmol) and DMAP (2 mg, 0.015 mmol) were dissolved in 10 mL of dry dimethylformamide. This mixture was stirred at rt for overnight. The crude mixture was purified by dialysis (MWCO 2000) for 24 h against 3 L water which was changed every 12 h. The dialyzed sample was frozen in liquid nitrogen and lyophilized. The resulting polymer was dissolved in DCM and filtered. This solution was concentrated and reprecipitated using diethylether to result in a pale yellow solid. The polymer was purified twice by dissolution-reprecipitation using DCM-ether and finally dried to obtain the Pt(IV) conjugated, PLA-Pt(IV). Yield of the purified polymer was 65%. This lyophilized product was dissolved in dichloromethane and filter to remove excess monosuccinato-Pt(IV). Solvent was then evaporated to get off white yellow solid product. Yield (26 mg, 68%). ¹H NMR (CDCl₃, 400 MHz): δ PPM, 7.90 (d, 4H), 7.55 (t, 4H), 7.25 (t, 4H), 7.08 (d, 4H), 6.86-6.96 (br m, 18H), 5.14 (t, 239H), 4.45-4.16 (m, 16H), 3.69 (br 6H), 3.37 (t, 2H), 2.73 (m, 4H), 2.47 (m, 12H), 2.28-2.33 (s, 12H), 2.04 (d, 9H), 1.72 (t, 2H), 1.56 (d, 851H). ¹³C NMR (CDCl₃, 100 MHz): δ 195.31, 175.11, 169.57, 139.27, 136.18, 131.25, 129.71, 126.12, 123.93, 123.10, 119.54, 117.71, 111.94, 110.15, 68.98, 16.62.

Synthesis of PLGA-Tris:

A mixture of PLGA-COOH (inherent viscosity d/L 0.15-0.25) (500 mg, 0.090 mmol) and NHS (12.37 mg, 0.107 mmol) in dry CH₂Cl₂ was stirred for 30 min at 0° C. A solution of DCC (20.34 mg, 0.098 mmol) in CH₂Cl₂ was added drop wise to the reaction mixture. The reaction was stirred from 0° C. to room temperature for 12 h. The precipitated N,N′-dicyclohexylurea (DCU) by-product was filtered off and the solution was evaporated using rotavap. This residue was dissolved in dry CH₂Cl₂. A solution of triethylamine (18.13 mg, 0.18 mmol) and Tris (22.0 mg, 0.18 mmol) in 10 mL DMF was added slowly to the above reaction mixture. This reaction mixture was kept at room temperature for 24 h with vigorous stirring. The solvent was evaporated to dryness. The residue was dissolved in CH₂Cl₂, filtered and precipitated with diethyl ether and methanol (CH₂Cl₂:MeOH:diethyl ether:1:5:4). This process was repeated 5 times. Yield, 190 mg, 37%. ¹H NMR (CDCl₃, 400 MHz): δ ppm 5.20 (m), 4.81 (m), 4.28 (br), 3.66 (s), 1.56 (d).

Synthesis of PLGA-Tris-Asp:

A mixture of PLGA-Tris (100 mg, 0.016 mmol) and DMAP (6.1 mg, 0.050 mmol) in dry CH₂Cl₂ was stirred for 30 min at room temperature. A solution of aspirin chloride (99.0 mg, 0.5 mmol) in CH₂Cl₂ was added drop wise to the reaction mixture. The reaction was stirred at room temperature for 24 h. The solvent was evaporated to dryness. The residue was dissolved in CH₂Cl₂, precipitated with diethyl ether and methanol (CH₂Cl₂:MeOH:diethyl ether: 1:5:4). This process was repeated 5 times. Yield, 94 mg, 90%. ¹H NMR (CDCl₃, 400 MHz): δ ppm 7.90 (d), 7.60 (t), 7.19 (t), 7.03 (d), 5.21 (m), 4.82 (m), 3.24 (s), 1.88 (s), 1.58 (m).

Synthesis of PLGA-Pamidronate Conjugate:

Sodium salt of pamidronate (25.0 mg, 0.0896) was dissolved in 10% aqueous acetic acid and the solution was frozen and lyophilized. Free carboxylic group of PLGA (500 mg, 0.0896 mmol) was activated by dissolving the polymer in 4 mL of 1:1 mixture of DMSO and dichloromethane. The solution was kept at 0° C. for 2 h, under stirring. Previously lyophilized pamidronate was dissolved in 1 mL of DMSO and added to the reaction mixture (Note: It may not be fully soluble but make suspension and add) which was stirred for 2 h at 0° C. then at rt for 8-12 h. Reaction mixture was concentrated and product was precipitated with diethyl ether:methanol (1:1). Yield, 450 mg, 87%. ¹H NMR (CDCl₃) ppm: 5.16-5.20 (m), 4.62-4.87 (m), 2.62-2.74 (t), 1.44-1.51 (m). ¹³C NMR (CDCl₃): δ 169.38, 166.34, 69.15, 66.80, 60.89, 40.48, 16.64.

Synthesis of Prednisone Mono-Succinate:

Prednisone (0.2 g, 0.558 mmol), DMAP (68.41 mg, 0.558 mmol) and succinic anhydride (55.83 g, 0.558 mmol) were dissolved in 60 mL of dichloromethane and left to react overnight under stirring at rt for 3 days. Then, the reaction mixture was quenched with 5 mL of water. Subsequently, the reaction mixture was diluted with 50 mL of dichloromethane and extracted with 10% aq. NaHSO₄ (3×20 mL) and brine. The organic phase was dried over MgSO₄ and the solvent was evaporated to dryness to obtain a white solid product. Yield 160 mg, 62% and second time 175 mg, 68%. ¹H NMR (CDCl₃, 400 MHz): δ PPM, 7.69 (d, 1H), 6.22 (d, 1H), 5.08 (d, H), 4.70 (d, 1H), 2.85 (d, 1H). 2.72 (m, 5H), 2.27-2.51 (m, 4H), 1.90-2.07 (m, 4H), 1.69 (m, 1H), 1.42 (s, 4H). 1.26 (m, 1H), 0.67 (s, 3H). ¹³C NMR (CDCl₃, 100 MHz): δ 208.96, 186.63, 171.73, 155.63, 127.47, 124.51, 89.56, 67.89, 60.15, 51.42, 49.58, 49.51, 42.43, 36.06, 34.93, 33.66, 32.23, 28.66, 28.52, 23.23, 18.73, 15.48.

Synthesis of Prednisone Acid Anhydride:

A suspension of monosuccinato-prednisone (200 mg, 0.436 mmol) in 15 mL of CH₂Cl₂ was prepared and a solution of 1,3-dicyclohexylcarbodiimide (DCC) (45 mg, 0.2183 mmol) in 5 mL of CH₂Cl₂ was added. The reaction mixture was stirred at room temperature for overnight. The urea DCC by product dicyclohexylurea (DCU) was filtered off in a glass filter and washed with a small amount of CH₂Cl₂. The solvent was evaporated and the resulting residue was taken up in EtOAc. Residual DCU was removed by filtering the resulting suspension through a glass filter. The filtrate was evaporated to give anhydride as white solid oil. Compound was used directly for next reaction. Yield 219 mg, 50%. ¹H NMR (CDCl₃, 400 MHz): δ PPM, 7.66 (d, 1H), 6.19 (d, 1H), 5.07 (d, 1H), 4.74 (d, 1H), 2.73-2.87 (m, 6H). 2.29-2.48 (m, 4H), 1.91-2.05 (m, 6H), 1.56-1.69 (m, 3H), 1.40 (s, 4H), 1.28 (m, 2H). 1.10 (m, 1H), 0.65 (s, 3H). ¹³C NMR (CDCl₃, 100 MHz): δ 190.35, 186.64, 171.20, 155.20, 127.41, 124.59, 113.29, 88.53, 68.03, 60.15, 51.47, 49.57, 42.36, 36.08, 33.66, 32.21, 30.18, 28.21, 24.80, 23.24, 18.78, 15.45.

Synthesis of Ac-[G-2]-(Pred)₄:

Ac-[G-2]-(OH)₄ (19.54 mg, 0.046 mmol) and DMAP (3.45 mg, 0.028 mmol) were dissolved in of CH₂Cl₂ in a 50 mL round bottom flask. The anhydride of monosuccinato-prednisone (219 mg, 0.243 mmol) was added slowly. The solution was stirred at room temperature overnight. The reaction was quenched with 4 mL of water under vigorous stirring, followed by dilution with 50 mL of CH₂Cl₂ and the solution was washed with and 10% of Na₂CO₃ (3×20 mL) and brine (10 mL). The organic phase was dried with MgSO₄, filtered, and concentrated to get pasty mass as product. %. ¹H NMR (CDCl₃, 400 MHz): δ PPM, 7.66 (d, 3H), 6.14 (d, 3H), 6.03 (s, 3H), 5.02 (d, 3H), 4.70 (d, 3H), 4.18 (m, 6H), 2.86 (m, 3H). 2.67 (m, 12H), 2.25-2.49 (m, 14H), 1.66-1.99 (m, 18H), 1.53 (m, 8H), 1.39 (s, 12H), 1.06-1.23 (m, 18H), 0.61 (s, 9H). ¹³C NMR (CDCl₃, 100 MHz): δ 209.58, 205.09, 186.89, 175.20, 172.02, 167.91, 157.69, 156.26, 127.24, 124.25, 88.43, 68.24, 60.01, 51.40, 49.50, 49.23, 42.57, 36.08, 34.61, 33.58, 32.29, 28.84, 28.71, 25.46, 24.80, 23.24, 18.79, 18.70, 15.38.

Synthesis of mono-succinato-Pt(IV):[di-ammino-dichloro-mono-succinatoplatinum (IV)]:

A mixture of di-ammino-dichloro-di-hydroxyplatinum(IV) {0.2 g, 0.59 mmol} and succinic anhydride (0.054 g, 0.531 mmol) in DMSO (16 mL) was stirred for 24 h. Solvent was then concentrated by lyophilization. The crude product was recrystallize through acetone and keeping at −20 C for 2 h followed by separation of the product as whitish yellow solid though centrifugation. Yield 160 mg (63%). 1H NMR (CDCl3, 400 MHz): δ PPM, 5.77-56.03 (broad m, 6H), 2.27-2.33 (m, 4H). 13C NMR (CDCl3, 300 MHz): δ 180.18, 174.52, 31.76, 30.68.

Preparation of NPs:

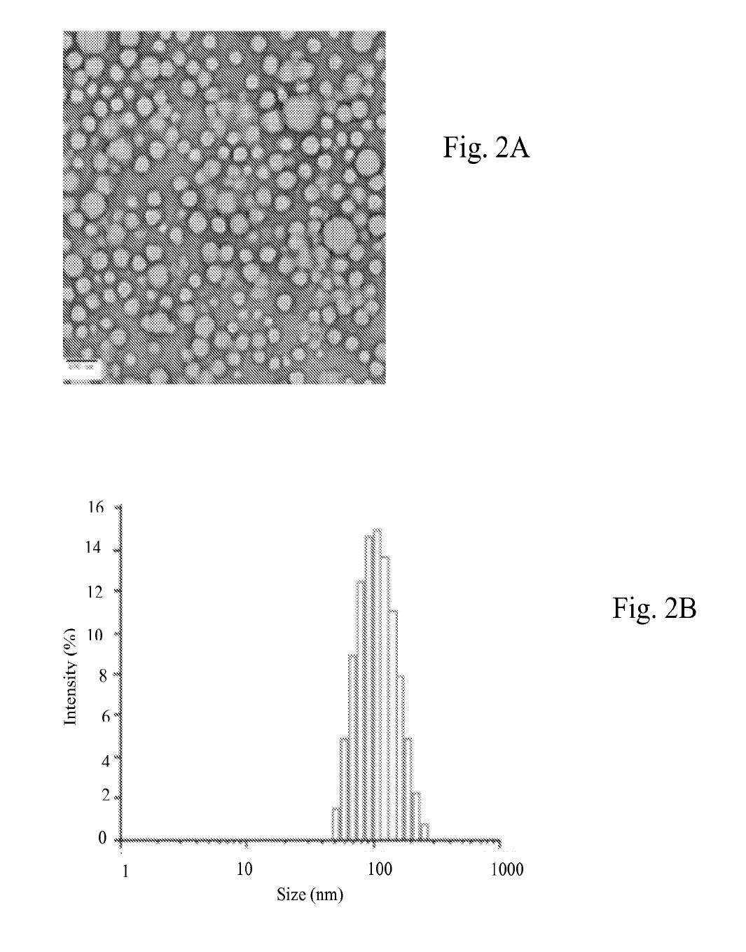

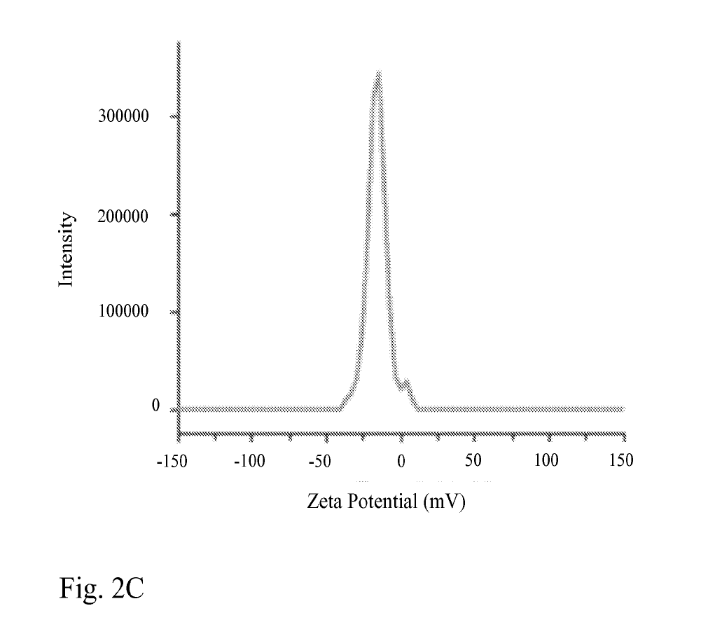

Combination therapeutic NP synthesized via self-assembly of PLGA-b-PEG-OH and (Aspirin)₄-PLA-[Pt(IV)prodrug]₃ polymer (FIG. 1), through a nanoprecipitation method. Dynamic light scattering (DLS) and transmission electron microscopy (TEM) were used to reveal the size and morphology of these NPs (FIGS. 2A and 2B), which was 106.2±1.9 nm. Zeta potential measurements showed that the NPs are negatively charged (FIG. 2C), having a zeta potential of −15.8±0.4 mV. Composition analysis of the NPs by inductively coupled plasma mass spectrometry (ICP-MS) for Pt indicated 2.4% loading and HPLC showed a loading of 3.0% for aspirin.

Anticancer Activity an In Vitro Study:

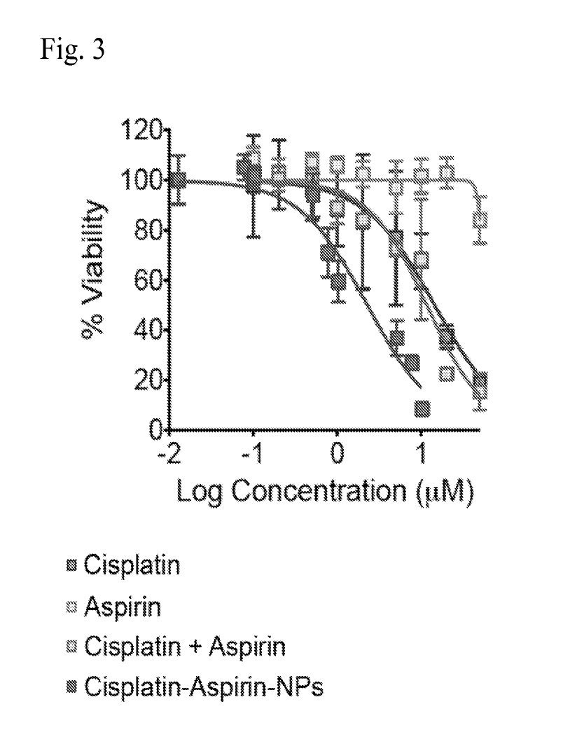

To demonstrate the combination therapeutic ability, in vitro anticancer activity of this platform was checked by using the MTT (3-(4,5-Dimethylthiazol-2-yl)-2,5-diphenyltetrazolium bromide) assay on human prostate cancer PC-3 cell line and compared with cisplatin, aspirin, and their combination in free formulation (incubation time=48 h; nanoparticle treatment=12 h.). The nanoparticle construct was found to be more effective in compared with the free drugs or their combination in the free formulations (FIG. 3). The IC₅₀ of the various constructs on PC-3 cells is presented in Table 1 below.

TABLE 1
Cytotoxic profile by MTT assay

| Therapeutic Construct | IC50 (μM) |
|---|---|
| Cisplatin | 14 |
| Aspirin | >100 |
| Free cisplatin + free aspirin | 12 |
| Cisplatin-aspirin-nanoparticles | 2.3 with respect to cisplatin |
| | 23 with respect to aspirin |

Summary:

A combinational therapeutic approach was developed to deliver chemotherapeutic and anti-inflammatory drugs for the treatment of CRPC. Aspirin functionalized alkyne dendron was synthesized and coupled with PLA-azide through click chemistry approach and this was finally decorated with Pt(IV) prodrugs. Blended NPs were synthesized using these polymers and characterized by DLS and TEM. Initial studies showed that the nanoparticles have significant anticancer effect on prostate cancer cells.

Other polymer-conjugated therapeutic agents or therapeutic agent-dendrons have been synthesized. Such compounds may be readily incorporated into nanoparticles.

Thus, embodiments of COMBINATION THERAPEUTIC NANOPARTICLES are disclosed. One skilled in the art will appreciate that the nanoparticles and methods described herein can be practiced with embodiments other than those disclosed. The disclosed embodiments are presented for purposes of illustration and not limitation.

1-7. (canceled)

8. A method for treating prostate cancer in a subject in need thereof, comprising:
administering a therapeutically effective amount of a nanoparticle comprising a chemotherapeutic agent and an anti-inflammatory agent to the subject.

9. A method according to claim 8, wherein the nanoparticle comprises a hydrophilic core and a hydrophilic layer surrounding the core.

10. A method according to claim 8, wherein the chemotherapeutic agent and the anti-inflammatory agent are attached to the core.

11. A method according to claim 8, wherein the chemotherapeutic agent is cisplatin.

12. A method according to claim 11, wherein the anti-inflammatory agent is aspirin.

13. A method according to claim 8, wherein the prostate cancer is castration resistant prostate cancer.

14. A use according to claim 8, wherein the nanoparticle is more cytotoxic to prostate cancer cells than a combination of a comparable dose of the free chemotherapeutic agent and the free anti-inflammatory agent.

15. A nanoparticle, comprising:
a hydrophobic nanoparticle core, wherein the hydrophobic polymer that forms at least a part of the core is selected from the group consisting of a polymer comprising polylactic acid (PLA) and a polymer comprising polylactic-co-glycolic acid (PLGA);
a hydrophilic layer surrounding the core, wherein the hydrophilic polymer moiety is attached to the core via a hydrophobic polymer moiety that forms at least a part of the core;
an anti-inflammatory agent attached to the core; and
a chemotherapeutic agent attached to the core.

16. A nanoparticle according to claim 15, wherein the anti-inflammatory agent is a corticosteroid or a non-steroidal anti-inflammatory drug.

17. A nanoparticle according to claim 15, the anti-inflammatory agent is aspirin.

18. A nanoparticle according to claim 15, the anti-inflammatory agent is prednisone.

19. A nanoparticle according to claim 15, further comprising a prostate cancer targeting moiety.

20. A nanoparticle according to claim 19, wherein the targeting moiety is a moiety configured to selectively bind to a prostate specific membrane antigen.

21. A nanoparticle according to claim 19, wherein the targeting moiety comprises a peptide having an amino acid sequence of WQPDTAHHWATL (SEQ ID NO:1) or a prostate specific membrane antigen binding fragment thereof.

22. A nanoparticle according to claim 15, wherein the anti-inflammatory agent is conjugated to a polymer forming at least a part of the hydrophobic core.

23. A nanoparticle according to claim 15, wherein the chemotherapeutic agent is conjugated to a polymer forming at least a part of the hydrophobic core.

24. A nanoparticle according to claim 23, wherein the polymer to which the anti-inflammatory agent is conjugated is the polymer to which the chemotherapeutic agent is conjugated.

25. A nanoparticle according to claim 15, further comprising an inhibitor of bone resorption.

26. A nanoparticle according claim 25, wherein the inhibitor of bone resorption is pamidronate.

27. (canceled)

28. A nanoparticle, comprising:
a hydrophobic nanoparticle core, wherein the hydrophobic polymer that forms at least a part of the core is selected from the group consisting of a polymer comprising polylactic acid (PLA) and a polymer comprising polylactic-co-glycolic acid (PLGA);
a hydrophilic layer surrounding the core, wherein the hydrophilic polymer moiety is attached to the core via a hydrophobic polymer moiety that forms at least a part of the core;
an inhibitor of bone resorption attached to the core; and
a chemotherapeutic agent attached to the core.

29. A compound selected from the group consisting of: 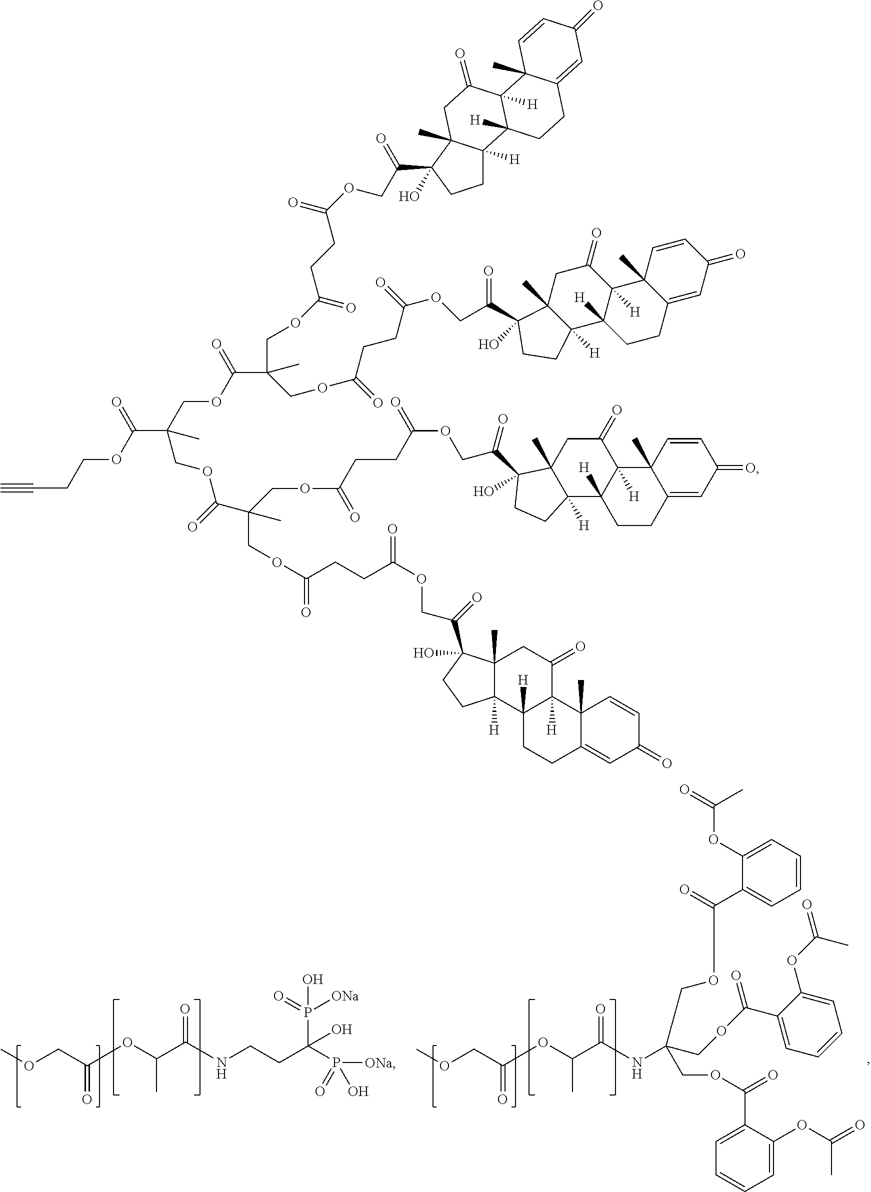 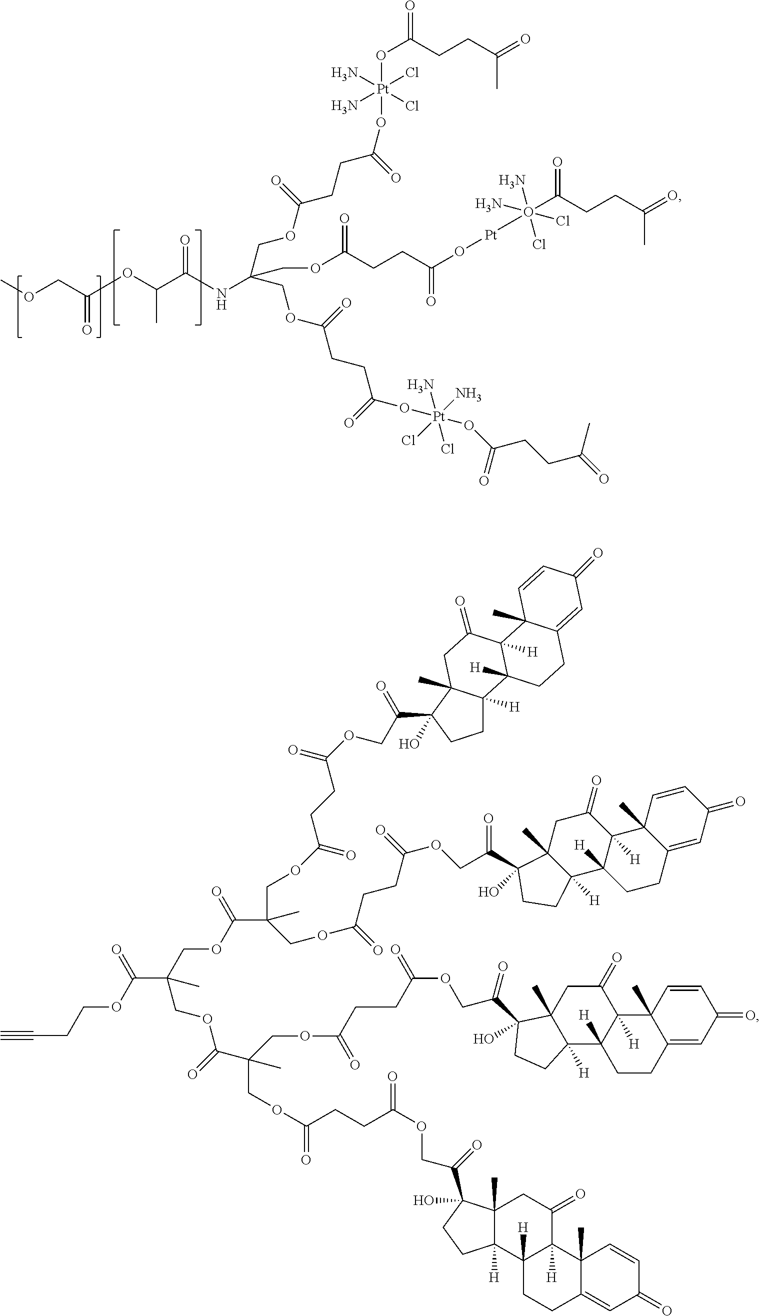 [chemical structure]
